(19) United States
(12) Patent Application Publication
(10) Pub. No.: US 2009/0326525 A1
(43) Pub. Date: Dec. 31, 2009

(54) LASER FIBER CAPILLARY APPARATUS AND METHOD

(76) Inventors: Jessica HIXON, Watertown, MA (US); Christopher L. OSKIN, Grafton, MA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/436,553

(22) Filed: May 6, 2009

Related U.S. Application Data
(60) Provisional application No. 61/075,830, filed on Jun. 26, 2008.

(51) Int. Cl. A61B 18/22; G02B 6/26

(52) U.S. Cl. 606/15; 385/38

(57) ABSTRACT

A method and an apparatus according to an embodiment of the invention includes a capillary for use in side-firing optical fibers. An outer surface of the capillary defines a recessed transmissive portion of the capillary. The area of the recessed transmissive portion can be a four-sided area or an area with a rounded boundary, for example. An optical-fiber-core end portion disposed within the capillary can include an end surface configured to redirect laser energy in a lateral direction and through the recessed transmissive portion of the capillary. The lateral direction can be substantially normal to the recessed transmissive portion of the capillary and offset from a longitudinal axis of the distal end portion of the core. The end surface of the core can be non-perpendicular to the longitudinal axis. In some embodiments, a multilayer dielectric coating can be disposed on the end surface of the core.

### RELATED APPLICATION

This application claims priority to and the benefit of U.S. Provisional Application No. 61/075,830, filed on Jun. 26, 2008, entitled “Laser Fiber Capillary Apparatus and Method,” which is incorporated herein by reference in its entirety.

### BACKGROUND

The invention relates generally to medical devices and more particularly to side-firing optical fibers and methods for using such devices.

Laser-based surgical procedures using side-firing optical fibers can provide a medical practitioner with more control when applying laser energy to the appropriate treatment area. Passing the distal end portion of the optical fiber through an endoscope during surgery, however, may damage, scratch, degrade, and/or deform the distal end portion of the optical fiber. To protect the optical-fiber end portion, a capillary and/or a metal cap or cannula, usually made of surgical grade stainless steel, can be placed over the optical-fiber end portion. Once the optical-fiber end portion is properly positioned for treatment, the laser energy can be applied to the target area.

During use of the device, a portion of the laser energy can leak from the optical-fiber end, reducing the efficiency with which laser energy is delivered to the treatment area and/or increasing overheating of the metal cap that is typically used to protect the optical fiber. Cooling of the device may be needed to operate at a safe temperature. In some instances, the overheating that can occur from the laser energy leakage can affect the mechanical and/or optical properties of the optical-fiber end portion, the capillary and/or the metal cap. In other instances, the overheating that can occur from the laser energy leakage can be sufficiently severe to damage the optical-fiber end portion, the capillary and/or the metal cap.

Overheating can also occur from the use of reflectors such as metallic reflectors or tips configured to redirect or bend an optical beam about 90 degrees from its original propagation path within the optical-fiber end portion based on total internal reflection (TIR). Because metallic reflectors do not reflect 100% of the optical beam, the energy associated with the non-reflected portion of the optical beam can be absorbed by the metallic reflector causing the metallic reflector to self heat. For TIR-based tips, a portion of the optical beam can leak through and heat up a protective metal cap positioned on a distal end of the tip. Furthermore, the glass capillary tubing that is typically used on the TIR-based tips can become damaged as tissue is ablated and impacts against the glass capillary tubing.

Thus, a need exists for optical-fiber end portions that can increase side-fired laser energy, increase device longevity, increase transmission efficiency, reduce overheating, and/or increase patient safety.

### SUMMARY

An apparatus includes a capillary for use in side-firing optical fibers. An outer surface of the capillary defines a recessed transmissive portion of the capillary. The recessed transmissive portion can be substantially flat. The area of the recessed transmissive portion can be a four-sided area or an area with a rounded boundary, for example. An optical-fiber-core end portion disposed within the capillary can include an end surface configured to redirect laser energy in a lateral direction and through the recessed transmissive portion of the capillary. The lateral direction can be substantially normal to the recessed transmissive portion of the capillary and offset from a longitudinal axis of the distal end portion of the optical fiber core. The end surface of the optical fiber core can be non-perpendicular to the longitudinal axis of the optical-fiber-core end portion. In some embodiments, a multilayer dielectric coating can be disposed on the end surface of the optical fiber core.

### BRIEF DESCRIPTION OF THE DRAWINGS

FIG. 1 is a schematic representation of a side-firing optical fiber system according to an embodiment.

FIG. 2 is a cross-sectional view of an optical-fiber distal end portion according to an embodiment.

FIG. 3A is a cross-sectional view of a side-firing optical fiber with an optically-transmissive capillary, according to an embodiment.

FIG. 3B is a cross-sectional view of a side-firing optical fiber with an optically-transmissive capillary and multilayer dielectric coating, according to an embodiment.

FIG. 4 is cross-sectional view of a side-firing optical fiber with a capillary and outer member, according to an embodiment.

FIGS. 5A-5B are side perspective views of a side-firing optical fiber with a capillary having a substantially flat surface, according to embodiments.

FIGS. 6A-6C are top perspective views of a side-firing optical fiber with a capillary having a substantially flat surface, according to embodiments.

FIGS. 7A-7B are cross-sectional views of a side-firing optical fiber with a capillary having a substantially flat surface and a rounded distal end, according to embodiments.

FIG. 7C is an end view taken along line G-G of FIG. 7A.

FIG. 8 is a perspective view of a side-firing optical fiber with a first member and a second member, according to an embodiment.

FIG. 9 is a cross-sectional view of a side-firing optical fiber with a first capillary and a second capillary, according to an embodiment.

FIGS. 10-11 are flow charts illustrating a method according to an embodiment.

### DETAILED DESCRIPTION

The devices and methods described herein are generally related to the use of side-firing optical fibers within the body of a patient. For example, the devices and methods can be used in treating symptoms related to an enlarged prostate gland, a condition known as Benign Prostatic Hyperplasia (BPH). BPH is a common condition in which the prostate becomes enlarged with aging. The prostate is a gland that is part of the male reproductive system. The prostate gland includes two lobes that are enclosed by an outer layer of tissue and is located below the bladder and surrounding the urethra, the canal through which urine passes out of the body. Prostate growth can occur in different types of tissue and can affect men differently. As a result of these differences, treatment varies in each case. No cure for BPH exists and once the prostate begins to enlarge, it often continues, unless medical treatment is initiated.

Patients who develop symptoms associated with BPH generally need some form of treatment. When the prostate gland is mildly enlarged, research studies indicate that early treatment may not be needed because the symptoms clear up without treatment in as many as one-third of cases. Instead of immediate treatment, regular checkups are recommended. Only if the condition presents a health risk or the symptoms result in major discomfort or inconvenience to the patient is treatment generally recommended. Current forms of treatment include drug treatment, minimally-invasive therapy, and surgical treatment. Drug treatment is not effective in all cases and a number of procedures have been developed to relieve BPH symptoms that are less invasive than conventional surgery.

While drug treatments and minimally-invasive procedures have proven helpful for some patients, many doctors still recommend surgical removal of the enlarged part of the prostate as the most appropriate long-term solution for patients with BPH. For the majority of cases that require surgery, a procedure known as Transurethral Resection of the Prostate (TURP) is used to relieve BPH symptoms. In this procedure, the medical practitioner inserts an instrument called a resectoscope into and through the urethra to remove the obstructing tissue. The resectoscope also provides irrigating fluids that carry away the removed tissue to the bladder.

More recently, laser-based surgical procedures employing side-firing optical fibers and high-power lasers have been used to remove obstructing prostate tissue. In these procedures, a doctor passes the optical fiber through the urethra using a cystoscope, a specialized endoscope with a small camera on the end, and then delivers multiple bursts of laser energy to destroy some of the enlarged prostate tissue and to shrink the size of the prostate. Patients who undergo laser surgery usually do not require overnight hospitalization and in most cases the catheter is removed the same day or the morning following the procedure. Generally, less bleeding occurs with laser surgery and recovery times tend to be shorter than those of traditional procedures such as TURP surgery.

A common laser-based surgical procedure is Holmium Laser Enucleation of the Prostate (HoLEP). In this procedure, a holmium:YAG (Ho:YAG) laser is used to remove obstructive prostate tissue. The Ho:YAG surgical laser is a solid-state, pulsed laser that emits light at a wavelength of approximately 2100 nm. This wavelength of light is particularly useful for tissue ablation as it is strongly absorbed by water. An advantage of Ho:YAG lasers is that they can be used for both tissue cutting and for coagulation. Another common laser surgery procedure is Holmium Laser Ablation of the Prostate (HoLAP), where a Ho:YAG laser is used to vaporize obstructive prostate tissue. The decision whether to use HoLAP or HoLEP is based primarily on the size of the prostate. For example, ablation may be preferred when the prostate is smaller than 60 cc (cubic centimeters). Laser-based surgical procedures, such as HoLAP and HoLEP, are becoming more preferable because they produce similar results to those obtained from TURP surgery while having fewer complications and requiring shorter hospital stay, shorter catheterization time, and shorter recovery time.

An optical fiber system as described herein can be used to transmit laser energy from a laser source to a target treatment area within a patient's body. The optical fiber system can include a laser source and an optical fiber. One end of the optical fiber can be coupled to the laser source while the other end of the optical fiber, the distal end portion (e.g., the end with a side-firing or laterally-firing portion), can be inserted into the patient's body to provide laser treatment. An angled or beveled end surface of the optical fiber core disposed within the capillary can redirect laser energy in a lateral direction for side-firing transmission of laser energy to the area of treatment. The angled end surface of the optical fiber core can include, for example, a multilayer dielectric coating. The multilayer dielectric coating can be configured to reflect a portion of the optical beam (e.g., laser beam) from the optical fiber that impinges on the end surface of the reflector at a less glancing angle and would not otherwise be totally internally reflected.

It is noted that, as used in this written description and the appended claims, the singular forms “a,” “an” and “the” include plural referents unless the context clearly dictates otherwise. Thus, for example, the term “a wavelength” is intended to mean a single wavelength or a combination of wavelengths. Furthermore, the words “proximal” and “distal” refer to direction closer to and away from, respectively, an operator (e.g., medical practitioner, medical practitioner, nurse, technician, etc.) who would insert the medical device into the patient, with the tip-end (i.e., distal end) of the device inserted inside a patient's body. Thus, for example, the optical fiber end inserted inside a patient's body would be the distal end of the optical fiber, while the optical fiber end outside a patient's body would be the proximal end of the optical fiber.

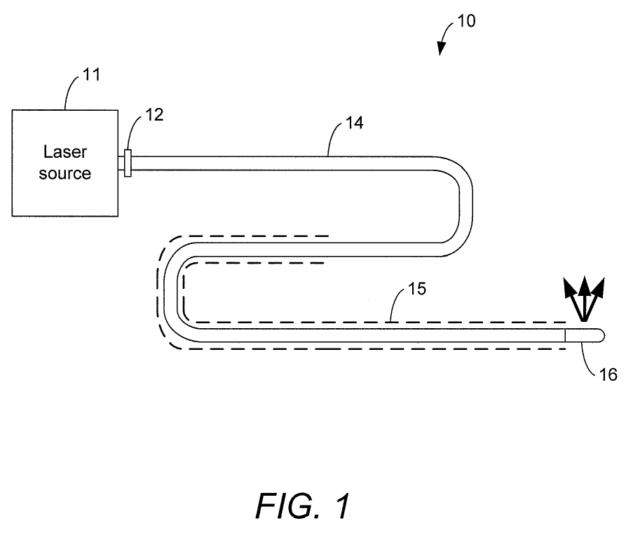

FIG. 1 is a schematic representation of a side-firing optical fiber system according to an embodiment. An optical fiber side-firing system 10 can include a laser source 11, an optical coupler 12, an optical fiber 14, and an optical-fiber distal end portion 16. The optical fiber side-firing system 10 also includes a suitable catheter or endoscope 15 for inserting the optical-fiber distal end portion 16 into a patient's body. The laser source 11 can include at least one laser that can be used to generate laser energy for surgical procedures. The laser source 11 can include a Ho:YAG laser, for example. The laser source 11 can include at least one of a neodymium-doped:YAG (Nd:YAG) laser, a semiconductor laser diode, or a potassium-titanyl phosphate crystal (KTP) laser, for other examples. In some embodiments, more than one laser can be included in the laser source 11 and more than one laser can be used during a surgical procedure. The laser source 11 can also have a processor that provides timing, wavelength, and/or power control of the laser. For example, the laser source 11 can include mechanisms for laser selection, filtering, temperature compensation, and/or Q-switching operations.

The optical fiber 14 can be coupled to the laser source 11 through the optical coupler 12. The optical coupler 12 can be an SMA connector, for example. The proximal end of the optical fiber 14 can be configured to receive laser energy from the laser source 11, and the distal end of the optical fiber 14 can be configured to output the laser energy through the optical-fiber distal end portion 16. The optical fiber 14 can include, for example, a core, one or more cladding layers about the core, a buffer layer about the cladding, and a jacket. The core can be made of a suitable material for the transmission of laser energy from the laser source 11. In some embodiments, when surgical procedures use wavelengths ranging from about 500 nm to about 2100 nm, the core can be made of silica with a low hydroxyl (OH⁻) ion residual concentration. An example of using low hydroxyl (low-OH) fibers in medical devices is described in U.S. Pat. No. 7,169,140 to Kume, the disclosure of which is incorporated herein by reference in its entirety. The cladding can be a single or a double cladding that can be made of a hard polymer or silica. The buffer can be made of a hard polymer such as Tefzel®, for example. When the optical fiber includes a jacket, the jacket can be made of Tefzel®, for example, or can be made of other polymers.

The endoscope 15 can define one or more lumens. In some embodiments, the endoscope 15 includes a single lumen that can receive therethrough various components such as the optical fiber 14. The endoscope 15 has a proximal end configured to receive the optical-fiber distal end portion 16 and a distal end configured to be inserted into a patient's body for positioning the optical-fiber distal end portion 16 in an appropriate location for a laser-based surgical procedure. For example, to relieve symptoms associated with BPH, the endoscope 15 can be used to place the optical-fiber distal end portion 16 at or near the enlarged portion of the prostate gland. In some instances, the endoscope 15 can be positioned at or near the enlarged portion of the prostate gland through the urethra. The endoscope 15 includes an elongate portion that can be flexible to allow the elongate portion to be maneuvered within the body. The endoscope 15 can also be configured to receive various medical devices or tools through one or more lumens of the endoscope, such as, for example, irrigation and/or suction devices, forceps, drills, snares, needles, etc. An example of such an endoscope with multiple lumens is described in U.S. Pat. No. 6,296,608 to Daniels et al., the disclosure of which is incorporated herein by reference in its entirety. In some embodiments, a fluid channel (not shown) is defined by the endoscope 15 and coupled at a proximal end to a fluid source (not shown). The fluid channel can be used to irrigate an interior of the patient's body during a laser-based surgical procedure. In some embodiments, an eyepiece (not shown) can be coupled to a proximal end portion of the endoscope 15, for example, and coupled to a proximal end portion of an optical fiber that can be disposed within a lumen of the endoscope 15. Such an embodiment allows a medical practitioner to view the interior of a patient's body through the eyepiece.

The optical-fiber distal end portion 16 can include one or more members, elements, or components that can individually or collectively operate to transmit laser energy in a lateral direction offset from a longitudinal axis or centerline of the distal end of the optical fiber core. In an embodiment, the optical-fiber distal end portion 16 can have a reflector or reflecting member with a multilayer dielectric coating on an angled surface for side-firing laser energy during a surgical procedure. Such a multilayer dielectric coating can be configured to have a high reflectance value (e.g., R>99.9%) at the laser operating wavelength and/or at the desired angle of incidence.

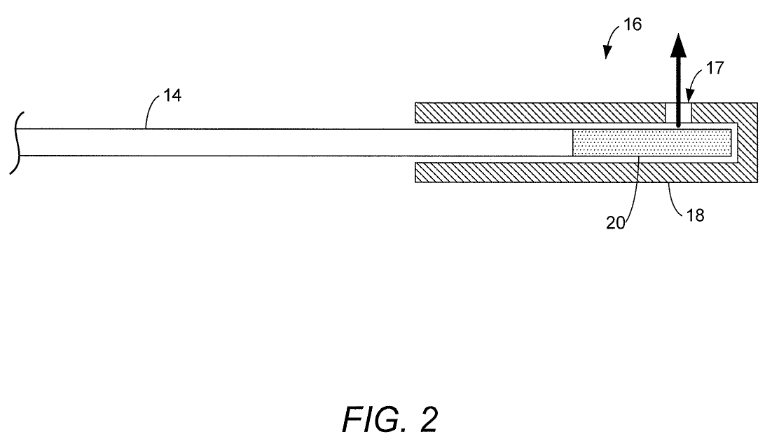

FIG. 2 is a cross-sectional view of an optical-fiber distal end portion according to an embodiment. The optical-fiber distal end portion 16 can include an inner portion 20 and surrounded by an outer portion 18. The outer portion 18 can include a high-profile member such as, for example, a metal or ceramic cover or cap. The cover or cap is generally made of surgical grade stainless steel or other materials with like properties. In some instances, it can be desirable to have the cap made of a ceramic material (e.g., alumina) because certain ceramics can offer stable characteristics at high-temperatures and/or have a high reflectance value at the laser operating wavelength. The outer portion 18 can provide protection to the optical-fiber distal end portion 16. In some embodiments, the outer portion 18 can include a low-profile cover (e.g., a coating or a sleeve).

The outer portion 18 can include a window or transmissive portion 17 through which laterally-redirected or side-fired laser energy can be transmitted for surgical treatment. For example, when the outer portion 18 is made of an opaque material, a window can be defined after removing at least a portion of the opaque material. In another example, when the outer portion 18 is made of an optically-transmissive material, laser energy can be transmitted or sent through the outer portion 18. In some embodiments, the optically-transmissive material can be treated thermally, optically, mechanically, and/or chemically to improve its structural and/or optical characteristics such that laser energy can be delivered more effectively to the target area. For example, the optically-transmissive material can be thermally treated during manufacturing using a CO₂ laser.

The inner portion 20 can include one or more members, components, and/or devices to redirect laser energy. For example, the inner portion 20 can include a capillary or capillary tube. The capillary can be made of, for example, at least one of silica, sapphire, and/or other like materials. In one embodiment, the inner portion 20 can include a distal end portion of the core of the optical fiber 14 disposed within a capillary. As described below in more detail, the inner portion 20 can also include reflecting members and/or mirrors that can be used to redirect laser energy to provide side-firing operations.

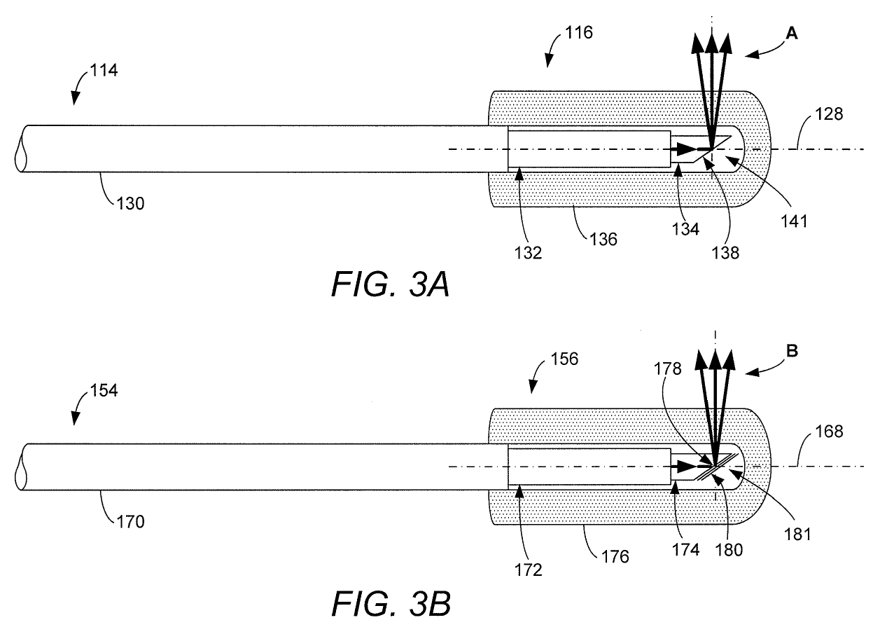

FIGS. 3A and 3B are each a cross-sectional view of side-firing optical fiber with an optically-transmissive capillary, according to an embodiment. FIG. 3A illustrates an optical-fiber distal end portion 116 having a capillary 136. In some embodiments, the capillary 136 can be made of an optically-transmissive material, for example. A distal end portion of a buffer layer 130, a distal end portion of a cladding layer 132, and/or an optical-fiber-core end portion 134 can be disposed within the capillary 136. In this regard, a region 141 within the capillary 136 can receive the distal end portion of the buffer layer 130, the distal end portion of a cladding layer 132, and/or the optical-fiber-core end portion 134. In this embodiment, the distal end portion of the buffer layer 130 is proximate to the distal end portion of the cladding layer 132, which is proximate to the optical-fiber-core end portion 134.

The optical-fiber-core end portion 134 can include a core-end surface 138 non-perpendicular to a longitudinal axis or centerline 128 of the optical-fiber-core end portion 134. In some instances, the core-end surface 138 can be referred to as an angled or beveled surface, for example. The core-end surface 138 can be configured to reflect laser energy (e.g., optical or laser beam) that is transmitted through the optical-fiber-core end portion 134 such that the laser energy is laterally redirected or side-fired. The core-end surface 138 can be used to redirect laser energy in a lateral direction offset from the longitudinal axis 128 of the optical-fiber-core end portion 134. The angle of the core-end surface 138 relative to the longitudinal axis can be determined based on at least one of several parameters. For example, the angle can be configured based on the wavelength of a laser energy, the exit or output location for the side-fired laser energy, and/or the optical properties of the optical-fiber-core end portion 134 and/or the capillary 136. Moreover, the optical properties of the region 141 can also be used in determining an appropriate angle for the core-end surface 138. In some instances, the angle of the core-end surface 138 can be determined based on a Brewster angle and/or on a total internal reflection (TIR) angle. By determining an appropriate angle for the core-end surface 138, the side-fired laser energy A can be transmitted in a lateral direction that is appropriate for laser-based surgical procedures.

In some instances, some of the laser energy transmitted through the optical-fiber-core end portion 134 is not laterally reflected at the core-end surface 138 and instead it is transmitted to the region 141 and then through the distal end of the capillary 136. This leakage or stray laser energy is thus transmitted in a direction that is substantially parallel to the longitudinal axis 128 of the optical-fiber-core end portion 134 and not in a laterally-redirected or side-fired direction. To minimize the amount of laser energy that is leaked in this manner, the core-end surface 138 can also include a reflective coating that operates to increase the efficiency with which the laser energy transmitted through the optical-fiber-core end portion 134 is laterally redirected for side-firing operations. Such an embodiment is discussed below in connection with FIG. 3B.

FIG. 3B is a cross-sectional view of an optical-fiber distal end portion 156 having a capillary 176. An optical-fiber-core end portion 174 having a core-end surface 178 can be disposed within the capillary 176. The core-end surface 178 is non-perpendicular to a longitudinal axis or centerline 168 of the optical-fiber-core end portion 174. The core-end surface 178 can include a multilayer dielectric coating 180. The multilayer dielectric coating 180 can be made of multiple dielectric layers that collectively operate to reflect laser energy. The multilayer dielectric coating 180 can include alternating layers of two or more materials each with a different dielectric constant. For example, the dielectric layers can be alternating layers of SiO₂ (silica) and TiO₂ (titanium dioxide or titania). In some embodiments, the multilayer dielectric coating 180 can be configured to operate as a ¼ wavelength mirror in which sets of two alternating layers are used and each layer from a set has an optical thickness that is ¼ the wavelength of the laser energy. Multiple deposition techniques, such as electron beam or ion beam deposition, for example, can be used to deposit the multilayer dielectric coating 180 on the core-end surface 178.

The multilayer dielectric coating 180 can be used to improve the efficiency with which a laser energy B is reflected at the core-end surface 138 when compared to other types of coated components, such as metallic mirrors or metallic coated glass mirrors, for example, which can absorb energy. The high reflectivity (e.g., high reflectance value at the laser operating wavelength) and low optical absorption of multilayer dielectric coatings can reduce the device operating temperature and/or reduce the amount of cooling that may be used to operate the device at a safe temperature.

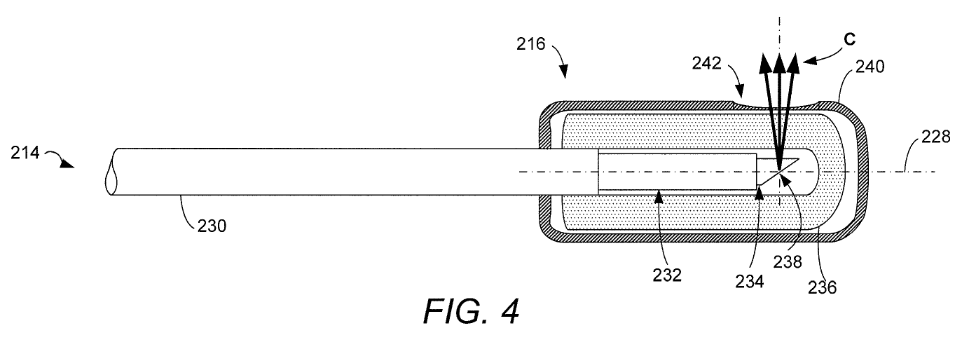

FIG. 4 is cross-sectional view of a side-firing optical fiber with a capillary and outer member, according to an embodiment. An optical-fiber distal end portion 216 can include a capillary 236 and an outer member 240. The outer member 240 can be disposed about the capillary 236. A distal end portion of a buffer layer 230, a distal end portion of a cladding layer 232, and/or an optical-fiber-core end portion 234 can be disposed within the capillary 236. The optical-fiber-core end portion 234 can include a core-end surface 238 non-perpendicular to a longitudinal axis or centerline 228 of the optical-fiber-core end portion 234. In this embodiment, the distal end portion of the buffer layer 230 is proximate to the distal end portion of the cladding layer 232, which is proximate to the optical-fiber-core end portion 234.

The outer member 240 can be a protective cover or cap made of a metal (e.g., stainless steel), a ceramic (e.g., alumina), or a polymer, for example. The outer member 240 can be coupled or affixed to a portion of the buffer layer 230 of an optical fiber 214. In one embodiment, the outer member 240 can be made of an optically-transmissive material. In another embodiment, the outer member 240 can be made of an optically-opaque material and can have an opening or window 242 such that laterally-redirected laser energy exits through the window 242. For example, a laser energy C transmitted through the optical fiber 214 can be reflected at the core-end surface 238 and can be transmitted through the capillary 236 before exiting the optical-fiber distal end portion 216 via the window 242 of the outer member 240. The core-end surface 238 can include a multilayer dielectric coating (not shown) having multiple dielectric layers that collectively operate to improve reflection efficiency of the laser energy C at the core-end surface 238.

In some instances, the outer member 240 can be a cap or sleeve having a proximal end opening that allows the outer member 240 to be disposed about the capillary 236 by sliding the outer member 240 over the capillary 236 such that a friction fit occurs. In other instances, the outer member 240 can be deposited about the capillary 236 such that the outer member 240 is at least partially in continuous and direct contact with the capillary 236. In yet another instance, the outer member 240 can be assembled about the capillary 236 such that a region (e.g., air gap) can occur between at least a portion of the outer surface of the capillary 236 and an inner surface of the outer member 240.

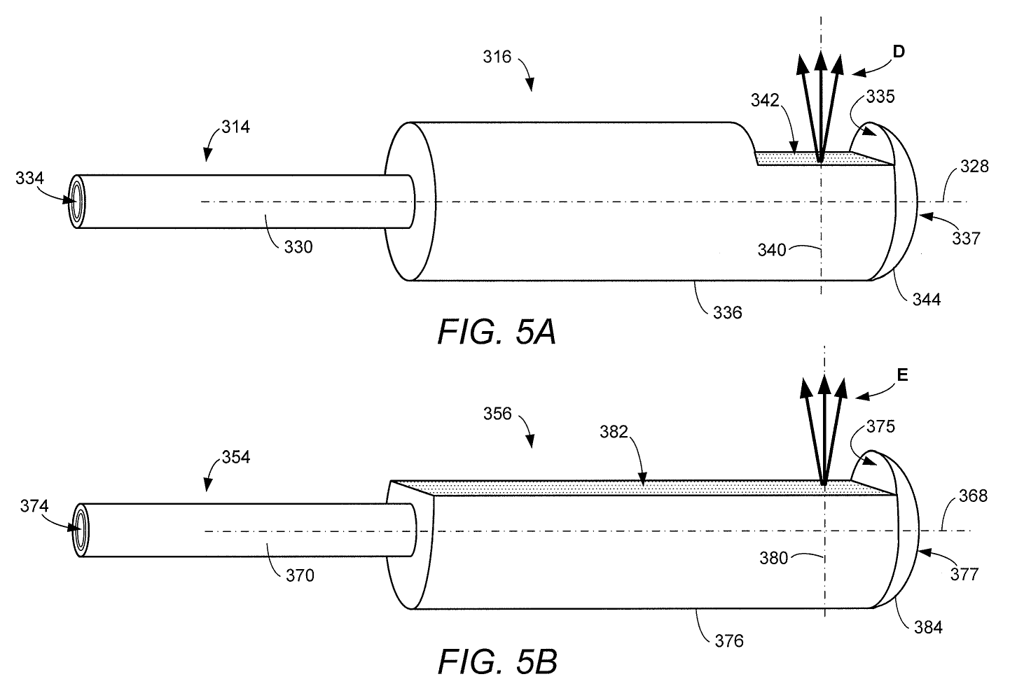

FIGS. 5A-5B each depicts a perspective view of a side-firing optical fiber with a capillary having a substantially flat surface, according to an embodiment. As shown in FIG. 5A, an optical-fiber distal end portion 316 can include a capillary 336 having a distal end portion of a buffer layer 330 and an end portion of the optical-fiber-core 334 disposed within the capillary 336. The end portion of the optical-fiber-core 334 disposed within the capillary 336 can include a core-end surface (not shown) non-perpendicular to a longitudinal axis or centerline 328 of the optical-fiber-core end portion 334. The core-end surface can be configured to reflect laser energy D that is transmitted longitudinally through an optical fiber 314 such that the laser energy D is laterally redirected and transmitted through a surface 342 of the capillary 336 during a laser-based surgical procedure.

The surface 342 can be referred to as a window, emissive portion, or transmissive portion of the capillary 336, for example. The surface 342 can be defined by an outer surface of the capillary 336. For example, the surface 342 can be produced by cutting and/or polishing a portion of the outer surface of the capillary 336, resulting in a substantially flat surface offset from the longitudinal axis 328. In one embodiment, the surface 342 can be substantially normal to a transversal axis 340 perpendicular to the longitudinal axis 328. In the example shown in FIG. 5A, the surface 342 has a distance or length that is less than the entire length of the capillary 336.

A member 344 can have a substantially flat proximal end 335 substantially perpendicular to the longitudinal axis 328 and a rounded distal end 337. The proximal end 335 of the member 344 can be coupled to the distal end of the capillary 336 by using a fusion process, for example. The shape and/or size of the member 344 can be configured to be inserted into a patient's body and/or to provide protection to the surface 342. The surface 342 is recessed, indented, or depressed between the outer surface of the capillary 336 and the outer edge of the member 344 so longitudinal movement through an endoscope, for example, does not damage, alter, and/or affect the surface 342. In some embodiments, members or components disposed on the surface 32, such as lenses, for example, which are recessed between the outer surface of the capillary 336 and the outer edge of the member 344 are protected during longitudinal movement through an endoscope. The end of the member 344 need not be a rounded end, for example, atraumatic tip ends having other shapes can be appropriate.

In another embodiment, the distal end of the capillary 336 can have a rounded end and a separate member having a rounded distal end need not be placed at the distal end of the capillary 336. In such embodiment, the surface 342 can have a length that is less than the entire length of the capillary.

As shown in FIG. 5B, an optical-fiber distal end portion 356 can include a capillary 376 having a distal end portion of a buffer layer 370 and an end portion of an optical-fiber-core 374 disposed within the capillary 376. The end portion of an optical-fiber-core 374 disposed within the capillary 376 can include a core-end surface (not shown) that is non-perpendicular to a longitudinal axis or centerline 368 of the optical-fiber-core end portion 374. The core-end surface can be configured to reflect laser energy E that is transmitted through an optical fiber 354 such that the laser energy E is laterally redirected and is transmitted through a surface 382 during a surgical procedure. The surface 382 can be a substantially flat surface offset from the longitudinal axis 368 and produced in a similar manner as the surface 342 disclosed in FIG. 5A. In the example shown in FIG. 5B, the surface 382 extends from the distal end of the capillary 376 to the proximal end of the capillary 376.

A member 384 can have a substantially flat proximal end 375 substantially perpendicular to the longitudinal axis 368 and a rounded distal end 377. The proximal end 375 of the member 384 can be coupled to the distal end of the capillary 356 through a fusion process, for example. The member 384 can be configured in a similar manner as the member 344 disclosed in FIG. 5A. The end of the member 384 need not be a rounded end, other end shapes can be appropriate.

In another embodiment, the distal end of the capillary 376 can have a rounded end and a separate member having a rounded distal end need not be placed at the distal end of the capillary 376. In such embodiment, the surface 382 can have a length between a rounded-distal-end portion of the capillary to a proximal end of the capillary.

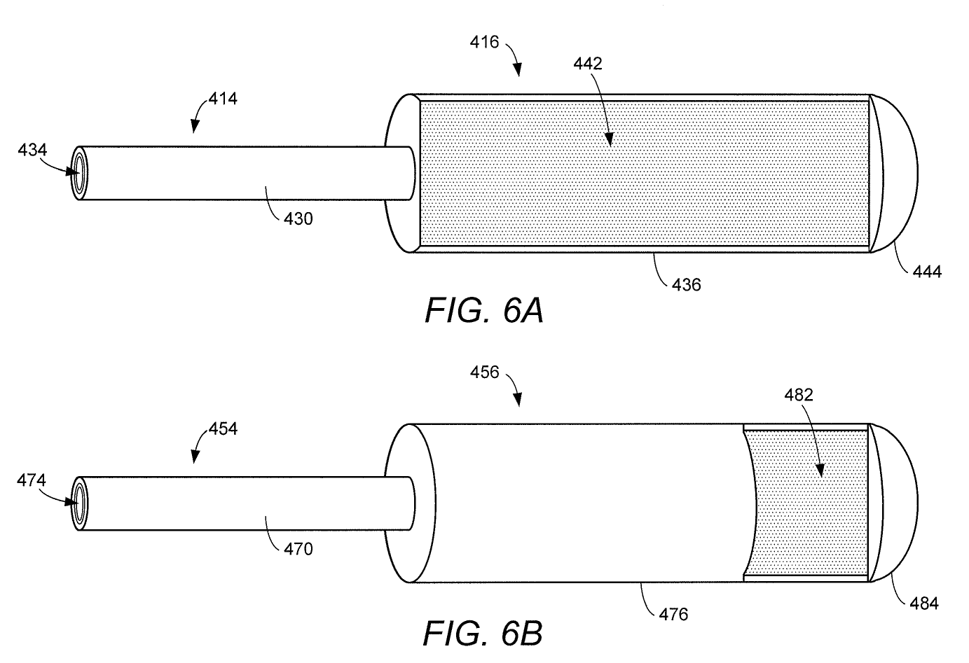

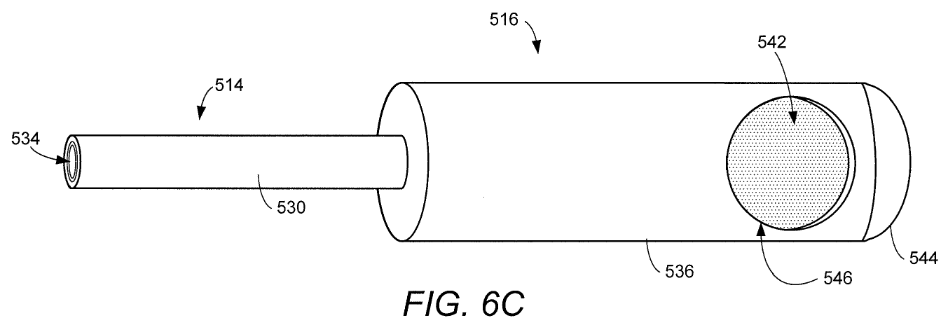

FIGS. 6A-6C each depicts a top perspective view of a side-firing optical fibers, according to embodiments. FIG. 6A, for example, is top perspective view of an optical-fiber distal end portion 416 with a capillary 436 having a surface 442 for transmission of laterally-redirected laser energy. A distal end portion of an optical fiber 414, including a distal end portion of a buffer layer 430 and an end portion of an optical-fiber-core 434, can be disposed within the capillary 436. The end portion of the optical-fiber-core 434 disposed within the capillary 436 can include a core-end surface (not shown) configured to redirect laser energy that is transmitted through the optical fiber 414 such that the laser energy is also transmitted through the surface 442 during a laser-based surgical procedure. A member 444 having a rounded distal end can be coupled to the distal end of the capillary 436 to facilitate insertion of the optical-fiber distal end portion 416 into a patient's body and/or to protect the surface 442 during insertion and/or positioning of the optical-fiber distal end portion 416 within an endoscope.

In the embodiment described in FIG. 6A, the surface 442 is shown having a substantially rectangular area and having a length between the proximal end of the capillary 436 and the distal end of the capillary 436. The area associated with the surface 442, however, need not be so limited. Other geometries can also be used for the area of the surface 442, such as an oval area or other polygonal areas, for example. In this regard, the shape and/or size of the area associated with the surface 442 can depend on the cutting and/or polishing operations used to achieve a desirable level of surface smoothness.

FIG. 6B illustrates a top perspective view of an optical-fiber distal end portion 456 including a capillary 476 having a surface 482. A distal end portion of an optical fiber 454, including a distal end portion of a buffer layer 470 and an end portion of an optical-fiber-core 474, can be disposed within the capillary 476. The end portion of the optical-fiber-core 474 disposed within the capillary 476 can include a core-end surface (not shown) configured to redirect laser energy that is transmitted through the optical fiber 454 such that the laser energy is also transmitted through the surface 482 during a laser-based surgical procedure. A member 484 having a rounded distal end can be coupled to the distal end of the capillary 476 to facilitate insertion of the optical-fiber distal end portion 456 into a patient's body and/or to protect the surface 482 during insertion and/or positioning of the optical-fiber distal end portion 456 within an endoscope.

In the embodiment described in FIG. 6B, the surface 482 is shown having a substantially square area and having a distance or length that is less than the entire length of the capillary 476. The area associated with the surface 482, however, need not be so limited. Other geometries can also be used for the area of the surface 482, such as an oval area, a circular area, or other polygonal areas, for example. For example, FIG. 6C illustrates a top perspective view of an optical-fiber distal end portion 516 including a capillary 536 having a surface 542. The surface 542 has a rounded area defined by an outer surface of the capillary 536 as indicated by a boundary 546. The shape and/or size of the areas associated with the surfaces 482 and 542 in FIGS. 6B and 6C, respectively, can depend on the cutting and/or polishing operations used to achieve a desirable level of surface smoothness.

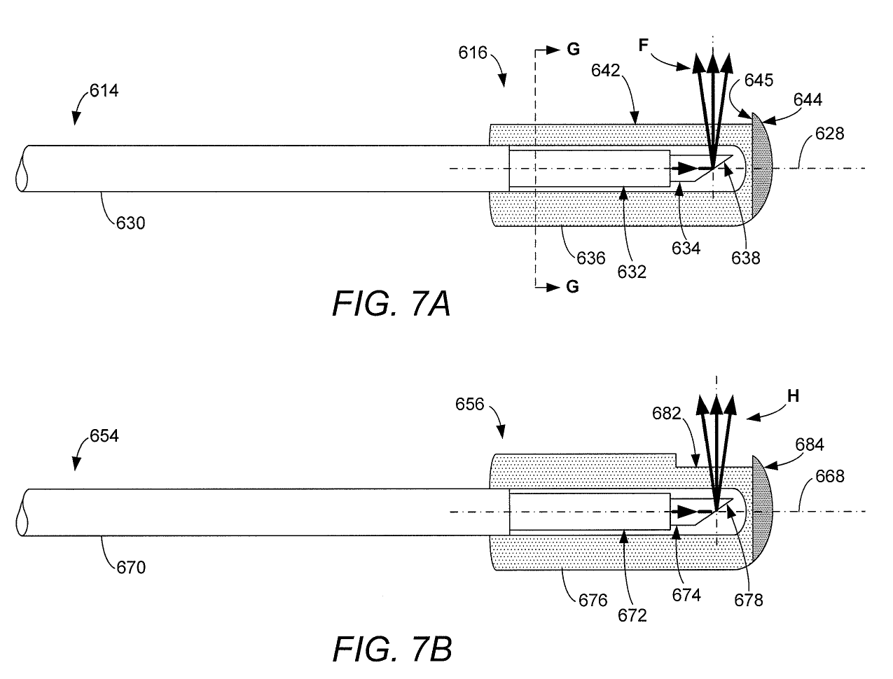

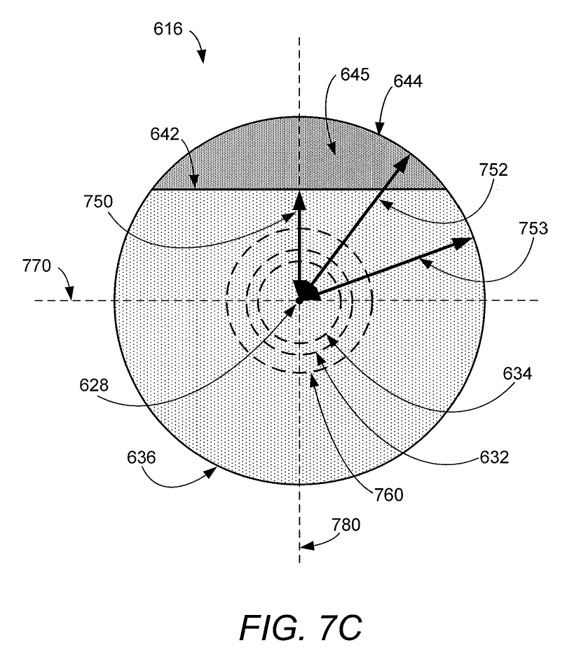

FIG. 7A illustrates a cross-sectional view of an optical-fiber distal end portion 616 with a capillary 636 having a surface 642 for transmission of laterally-redirected laser energy F. The capillary 636 can be made of an optically-transmissive material, for example. A distal end portion of a buffer layer 630, a distal end portion of a cladding layer 632, and/or an optical-fiber-core end portion 634 can be disposed within the capillary 636. In this embodiment, the distal end portion of the buffer layer 630 is proximate to the distal end portion of the cladding layer 632, which is proximate to the optical-fiber-core end portion 634. The optical-fiber-core end portion 634 can include a core-end surface 638 configured to redirect laser energy F in a lateral direction offset from a longitudinal axis or centerline 628 of the optical-fiber-core end portion 634.

The surface 642 can be defined by an outer surface of the capillary 636 and can be produced by cutting and/or polishing a portion of the outer surface of the capillary 636, resulting in a substantially flat surface offset from the longitudinal axis 628. In the example shown in FIG. 7A, the surface 642 has a length between the distal end of the capillary 636 and the proximal end of the capillary 636. A proximal end 645 of a member 644 having a rounded distal end can be coupled (e.g., fused) to the distal end of the capillary 636.

FIGS. 5A-6C illustrate a recessed, indented, or depressed substantially flat surface defined on a capillary and having a distal end edge or a profile such that the surface is protected during assembly and/or operation. For example, the substantially flat surface is protected from damage and/or scratches during assembly when a cap or sleeve (not shown) is slideably disposed about the capillary. In another example, the substantially flat surface is protected from damage and/or scratches during insertion and/or positioning within an endoscope.

FIG. 7B illustrates a cross-sectional view of an optical-fiber distal end portion 656 with a capillary 676 having a surface 682 for transmission of laterally-redirected laser energy H. A distal end portion of a buffer layer 670, a distal end portion of a cladding layer 672, and/or an optical-fiber-core end portion 674 can be disposed within the capillary 676. The optical-fiber-core end portion 674 can include a core-end surface 678 configured to redirect laser energy H in a lateral direction offset from a longitudinal axis or centerline 668 of the optical-fiber-core end portion 674.

The surface 682 can be defined by an outer surface of the capillary 676 and can be produced by cutting and/or polishing a portion of the outer surface of the capillary 676, resulting in a substantially flat surface offset from the longitudinal axis 668. In the example shown in FIG. 7B, the surface 682 has a distance or length that is partially the length of the capillary 676. A member 684 having a rounded distal end can be coupled (e.g., fused) to the distal end of the capillary 676.

FIG. 7C is an end view taken along line G-G of FIG. 7A. FIG. 7C shows a distal portion of the optical-fiber distal end portion 616, member 644, the capillary 636, the distal end portion of the cladding layer 632, the optical-fiber-core end portion 634, and an inner surface 760 of the capillary 636 that defines an inner region of the capillary 636. A length 750 along a plane 780 can be a distance between the surface 642 and the centerline 628. A length 752 can be a distance between an outer surface of a proximal end of the member 644 and the centerline 628. A length 753 can be a distance between an outer surface of the capillary 636 and the centerline 628. The centerline 628 can be substantially parallel to a plane 770 that is orthogonal to the plane 780. Because the length 750 is shorter than the lengths 752 and 753, the surface 642 can be recessed or indented such that the proximal end of the member 644 and the outer surface of the capillary 636 can provide protection to the surface 642 from damage, scratches, degradation, and/or deformation when the optical-fiber distal end portion 616 is inserted into and/or passed through an endoscope without the need to use a protective cover or cap. In this regard, the profile or size (e.g., radius, diameter) of the proximal end 645 of the member 644 and/or the capillary 636 can be similar to that of a metal cap typically used to protect an optical fiber in other embodiments.

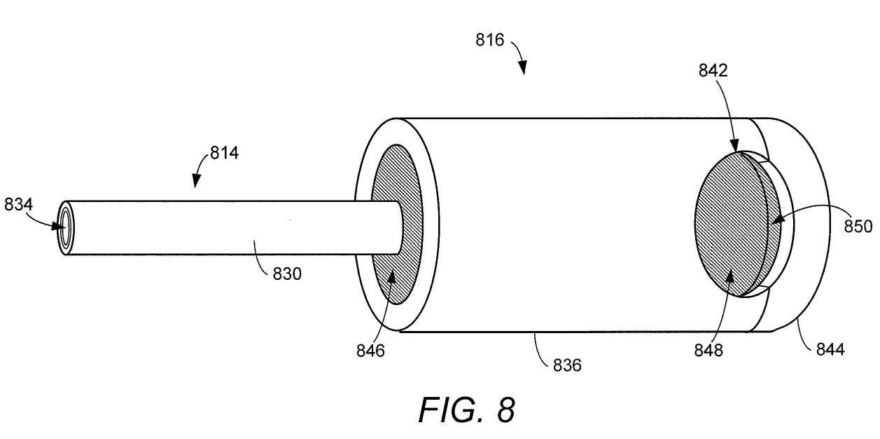

FIG. 8 illustrates a perspective view of an optical-fiber distal end portion 816 having a first member 846 and a second member 836. The first member 846 can be a capillary, for example, which can be made of an optically-transmissive material. A distal end portion of an optical fiber 814, including a distal end portion of a buffer layer 830 and an end portion of an optical-fiber-core 834, can be disposed within the first member 846. The end portion of the optical-fiber-core 834 disposed within the first member 846 can include a core-end surface (not shown) configured to reflect laser energy transmitted longitudinally through the optical fiber 814 such that the laser energy is laterally redirected and transmitted through a transmissive portion 848 of the first member 846 during a laser-based surgical procedure. In some instances, the transmissive portion 848 can be referred to as a window or emissive portion of the first member 846, for example. In some embodiments, the first member 846 can be coupled to an first end member 850. The first member 846 and the first end member 850 can be coupled by, for example, a process in which a distal end of the first member 846 is fused to a proximal end of the end member 850.

The second member 836 can be disposed about the first member 846. In this regard, the second member 836 can protects to the first member 846 during insertion and/or positioning of the optical-fiber distal end portion 816 within an endoscope. The second member 836 can be a capillary, for example. In one embodiment, the second member 836 can be made of an optically-opaque material. In this regard, an opening 842 can be defined by an outer surface of the second member 836 such that laser energy transmitted through the transmissive portion 848 of the first member 846 is also transmitted through the opening 842 of the second member 836. The opening 842 can protect the transmissive portion 848 of the first member 846 because the transmissive portion 848 is recessed or indented with respect to the opening 842.

In some embodiments, the second member 836 can be coupled to a second end member 844. The second member 836 and the second end member 844 can be coupled by, for example, a process in which a distal end of the second member 836 is fused to a proximal end of the second end member 844. The second end member 844 can be configured as an atraumatic tip to be inserted into a patient's body.

In the example shown in FIG. 8, a portion of the opening 842 can be defined by the outer surface of the second member 836 and a remaining portion of the opening 842 can be defined by an outer surface of the second end member 844. In this example, the opening 842 is shown as having a rounded boundary defining a circular opening, however, other geometries can also be used, such as an oval opening or a square opening, for example.

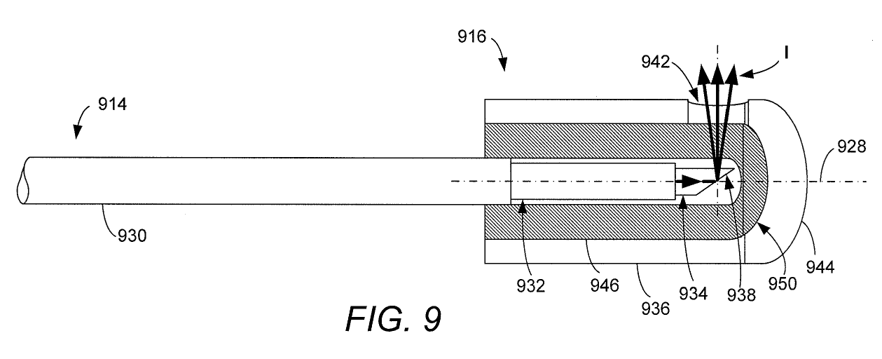

FIG. 9 is a cross-sectional view of an optical-fiber distal end portion 916 having a first capillary 946 and a second capillary 936. The second capillary 936 can be disposed about the first capillary 946. A distal end portion of an optical fiber 914, including a distal end portion of a buffer layer 930, a distal end portion of a cladding layer 932, and an optical-fiber-core end portion 934, can be disposed within the first capillary 946. The optical-fiber-core end portion 934 can include a core-end surface 938 non-perpendicular to a longitudinal axis or centerline 928 of the optical-fiber-core end portion 934.

In one embodiment, the first capillary 946 can be made of an optically-transmissive material and the second capillary 936 can be made of an optically-opaque material. In this regard, an opening 942 of the second capillary 936 can be defined on the outer surface of the second capillary 936 to allow laterally-redirected laser energy to exit through the opening 942. For example, a laser energy I transmitted longitudinally through the optical fiber 914 can be reflected at the core-end surface 938 and be further transmitted through the first capillary 946 before exiting the optical-fiber distal end portion 916 via the opening 942 of the second capillary 936. The core-end surface 938 can include a multilayer dielectric coating (not shown) having multiple dielectric layers that collectively operate to improve the reflection efficiency of the laser energy I at the core-end surface 938.

In the example shown in FIG. 9, a proximal end of a first member 950 can be coupled to a distal end of the first capillary 946, and a proximal end of a second member 944 can be coupled to a distal end of the second capillary 936. In this example, a portion of the opening 942 can be defined by the outer surface of the second capillary 936 and a remaining portion of the opening 942 can be defined by an outer surface of the second member 944. The second member 944 can be configured to be at least partially inserted into the patient's body during a laser-based surgical procedure.

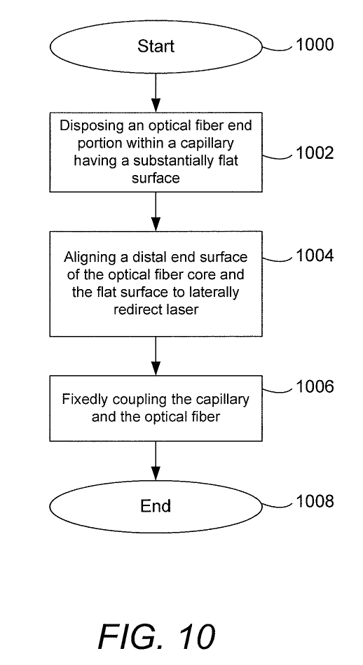

FIG. 10 is a flow chart illustrating a method for manufacturing a side-firing optical fiber, according to an embodiment. At 1002, after start 1000, a distal end portion of an optical fiber is disposed within an optically-transmissive capillary. The distal end portion of the optical fiber includes an optical-fiber-core end portion having a core-end surface configured to redirect laser energy in a lateral direction. In some embodiments, a multilayer dielectric coating is disposed on the core-end surface before step 1002. The capillary includes a substantially flat surface through which laterally-redirected laser energy can be transmitted. The substantially flat surface can have a length corresponding to or less than the length of the capillary. The substantially flat surface can have an area defined by a rounded (e.g., circular, oval) boundary or by a boundary having multiple sides (e.g., four-sided boundary). The side-firing optical fiber can include a member coupled to the distal end of the capillary.

At 1004, the substantially flat surface of the capillary and the core-end surface of the optical-fiber-core end portion are aligned such that in operation, laser energy redirected at the core-end surface is transmitted through the optically-transmissive capillary and exits via the substantially flat surface of the capillary. At 1006, the proximal end of the capillary can be fixedly coupled to the optical fiber (e.g., the outer surface of the optical fiber buffer). After 1006, the method can proceed to end 1008. An additional step can include, for example, adding a cap about the capillary. The cap can include an atraumatic tip, for example.

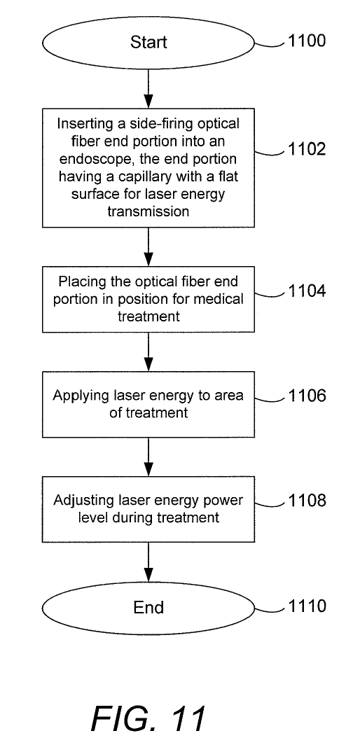

FIG. 11 is a flow chart illustrating a method of using an optical fiber side-firing system, according to another embodiment. At 1102, after start 1100, an optical-fiber distal end portion can be inserted within an inner portion or lumen of an endoscope. The optical-fiber distal end portion includes a capillary having an optical-fiber-core end portion. The optical-fiber-core end portion can have a distal end surface non-perpendicular to a longitudinal axis of the optical-fiber-core end portion. The distal end surface is configured to redirect laser energy in a lateral direction. In some embodiments, a multilayer dielectric coating is disposed on the distal end surface. At 1104, the endoscope can be at least partially inserted into the patient's body during a laser-based surgical procedure. Once inserted into the patient's body, the endoscope can be used to place or position the optical-fiber distal end portion at or near the area of treatment. For example, during laser-based surgical procedures to treat BPH, the endoscope can be positioned at or near the enlarged portion of the prostate gland through the urethra. At 1106, laser energy (e.g., laser or optical beam) from a laser source can be transmitted through the optical fiber such that laser energy is side-fired or laterally redirected to the treatment area. At 1108, a side-firing optical fiber system is used to dynamically and/or automatically control a laser energy power level during the laser-based surgical procedure. After 1108, the method can proceed to end 1110.

A recessed, indented, or depressed substantially flat surface having a distal end edge or a profile that protects the surface can result in reduced damage to the surface during assembly and/or operation. For example, the substantially flat surface can be protected from damage, scratches, degradation, and/or deformation during assembly of a side-firing optical fibbed end when a cap or sleeve is slidably disposed about the capillary. In another example, the substantially flat surface can be protected from damage, scratches, degradation, and/or deformation during insertion and/or positioning of the capillary within an endoscope.

### CONCLUSION

While various embodiments have been described above, it should be understood that they have been presented by way of example only, and not limitation. For example, the optical fiber side-firing system described herein can include various combinations and/or sub-combinations of the components and/or features of the different embodiments described. Although described with reference to use for treatment of symptoms related to BPH, it should be understood that the optical fiber side-firing system and the side-firing optical fibers, as well as the methods of using the optical fiber side-firing system and the side-firing optical fibers can be used in the treatment of other conditions.

Embodiments of a side-firing optical fiber can also be provided without the optical fiber side-firing system described herein. For example, a side-firing optical fiber can be configured to be used with other laser sources, endoscopes, etc., not specifically described herein. A side-firing optical fiber can have a variety of different shapes and sizes than as illustrated and described herein. A side-firing optical fiber can also include other features and/or components such as, for example, lenses and/or filters. The other features and/or components can be disposed on, for example, the substantially flat surface.

In one embodiment, an apparatus can include a capillary and an optical fiber. The capillary can have a recessed transmissive portion. The recessed transmissive portion of the capillary can be offset from a centerline of the capillary. The optical fiber can have a core. A distal end portion of the core can be disposed within the capillary. The distal end portion of the core can have a surface non-perpendicular to a longitudinal axis of the distal end portion of the core. The surface of the distal end portion of the core can be configured to redirect laser energy in a lateral direction offset from the longitudinal axis and through the recessed transmissive portion of the capillary. The lateral direction can be substantially normal to the recessed transmissive portion of the capillary. The optical fiber can include a buffer layer. The capillary can be fixedly coupled to the buffer layer of the optical fiber such that the buffer layer of the optical fiber is between the core of the optical fiber and the capillary.

An outer surface of the capillary can define the recessed transmissive portion of the capillary. The recessed transmissive portion of the capillary can be substantially flat. The recessed transmissive portion of the capillary can include a four-sided surface area or an area with a rounded boundary, for example.

A distance from the recessed transmissive portion of the capillary to the centerline of the capillary in a direction normal to the recessed transmissive portion of the capillary is shorter than a distance from the outer surface of the capillary to the centerline of the capillary.

In another embodiment, the apparatus can further include a multilayer dielectric coating disposed on the surface of the distal end portion of the core. The multilayer dielectric coating can include multiple layers having a first set of layers with an index of refraction and a second set of layers with an index of refraction different than the index of refraction of the first set of layers. The multiple layers of the multilayer dielectric coating can be alternating layers from the first set of layers and the second set of layers. In yet another embodiment, the apparatus can include a member having a rounded end coupled to a distal end of the capillary. The rounded end of the member can be configured to be inserted into a patient's body.

In one embodiment, an apparatus can include a first member and a second member. The first member can have a distal end configured to be inserted into a patient's body. An outer surface of the first member can define a recessed surface. The recessed surface can be offset from a centerline of the first member. The recessed surface of the first member can be substantially flat. The recessed surface of the first member can include an optically-transmissive portion of the first member. The first member can be, for example, a capillary tube.

The second member can have a distal end surface non-perpendicular to a longitudinal axis of the distal end portion of the second member. The distal end surface of the second member can be disposed within the first member. The distal end surface of the second member can be configured to redirect laser energy in a lateral direction offset from the longitudinal axis and through the recessed surface of the first member. The lateral direction can be substantially normal to the recessed surface of the first member. The second member can include an optical fiber core. The distal end surface of the second member can be a distal end surface of the optical fiber core. In another embodiment, the apparatus can further include a multilayer dielectric coating disposed on the distal end surface of the second member.

In one embodiment, an apparatus can include a first capillary, a second capillary, and an optical fiber core. The first capillary can have a transmissive portion. In one example, the first capillary can be made of an optically-transmissive material. The second capillary can have a transmissive portion. At least a portion of the first capillary can be disposed within the second capillary. The transmissive portion of the second capillary is at least partially aligned with the transmissive portion of the first capillary. An outer surface of the second capillary can define an opening such that the transmissive portion of the second capillary includes the opening. The first capillary and the second capillary can be fixedly coupled.

The optical fiber can have a core. A distal end portion of the core can be disposed within the first capillary. A distal end of the core can have a surface non-perpendicular to a longitudinal axis of the distal end portion of the core. The surface of the distal end of the core can be configured to redirect laser energy in a lateral direction offset from the longitudinal axis and through the transmissive portion of the first capillary and the transmissive portion of the second capillary. The optical fiber can include a buffer layer such that the first capillary can be fixedly coupled to the buffer layer of the optical fiber. In another embodiment, the apparatus can further include a multilayer dielectric coating disposed on the surface of the distal end of the core.

In one embodiment, an apparatus can include a capillary and an optical fiber core. The capillary can have a distal end configured to be inserted into a patient's body. An outer surface of the capillary can define a recessed portion. The recessed portion of the capillary can be offset from a centerline of the capillary. The recessed portion of the capillary can be substantially flat. The outer surface of the capillary can be is polished to produce the recessed portion of the capillary. An outer diameter of the capillary can be less than a diameter of a urethra, for example.

The optical fiber core can have a distal end surface disposed within the capillary. The distal end surface of the optical fiber core and the recessed portion of the capillary can be aligned such that laser energy is redirected in a lateral direction offset from a longitudinal axis of a distal end portion of the capillary and through the recessed portion of the capillary.

In another embodiment, the apparatus can further include a member having a rounded end coupled to a distal end of the capillary. The rounded end of the member can be configured to be inserted into a patient's body. In yet another embodiment, the apparatus can further include a multilayer dielectric coating disposed on the distal end surface of the optical fiber core.

In one embodiment, a method can include inserting a distal end portion of a first member into a patient's body. The first member can have an outer surface that defines a recessed surface. The first member can have a second member disposed within the first member. The second member can be configured to redirect laser energy in a lateral direction offset from a longitudinal axis of a distal end portion of the first member. The second member can include a distal end portion of an optical fiber core. A distal end of the optical fiber core can have a surface non-perpendicular to the longitudinal axis.

After the inserting of the distal end portion of the first member into the patient's body, the method can include activating a laser source to transmit laser energy to the patient's body, the transmitted laser energy passing through the recessed surface of the first member. The inserting of the distal end portion of the first member can include inserting the distal end portion of the first member into a urethra. The method can further include adjusting a power level of the laser energy transmitted to the patient's body.

What is claimed is:

1. An apparatus, comprising:
a capillary having a recessed transmissive portion, the recessed transmissive portion of the capillary being offset from a centerline of the capillary; and
an optical fiber having a core, a distal end portion of the core being disposed within the capillary, the distal end portion of the core having a surface non-perpendicular to a longitudinal axis of the distal end portion of the core, the surface of the distal end portion of the core being configured to redirect laser energy in a lateral direction offset from the longitudinal axis and through the recessed transmissive portion of the capillary.

2. The apparatus of claim 1, wherein an outer surface of the capillary defines the recessed transmissive portion of the capillary.

3. The apparatus of claim 1, wherein the lateral direction is substantially normal to the recessed transmissive portion of the capillary.

4. The apparatus of claim 1, wherein at least one of the recessed transmissive portion is substantially flat or the recessed transmissive portion of the capillary includes a four-sided surface area.

5. The apparatus of claim 1, wherein the recessed transmissive portion of the capillary includes an area with a rounded boundary.

6. The apparatus of claim 1, further comprising a multilayer dielectric coating disposed on the surface of the distal end portion of the core.

7. The apparatus of claim 1, further comprising a multilayer dielectric coating disposed on the surface of the distal end portion of the core, the multilayer dielectric coating including a plurality of layers having a first set of layers with an index of refraction and a second set of layers with an index of refraction different than the index of refraction of the first set of layers, the plurality of layers alternating layers from the first set of layers and the second set of layers.

8. The apparatus of claim 1, further comprising a member having a rounded end coupled to a distal end of the capillary, the rounded end configured to be inserted into a patient's body.

9. The apparatus of claim 1, wherein the optical fiber includes a buffer layer, the capillary being fixedly coupled to the buffer layer, the buffer layer of the optical fiber being between the core of the optical fiber and the capillary.

10. The apparatus of claim 1, wherein a distance from the recessed transmissive portion of the capillary to the centerline of the capillary in a direction normal to the recessed transmissive portion of the capillary is shorter than a distance from the outer surface of the capillary to the centerline of the capillary.

11. An apparatus, comprising:
a first capillary having a transmissive portion;
a second capillary having a transmissive portion, at least a portion of the first capillary being disposed within the second capillary, the transmissive portion of the second capillary at least partially aligned with the transmissive portion of the first capillary; and
an optical fiber having a core, a distal end portion of the core being disposed within the first capillary, a distal end of the core having a surface non-perpendicular to a longitudinal axis of the distal end portion of the core, the surface of the distal end of the core being configured to redirect laser energy in a lateral direction offset from the longitudinal axis and through the transmissive portion of the first capillary and the transmissive portion of the second capillary.

12. The apparatus of claim 11, wherein an outer surface of the second capillary defines an opening, the transmissive portion of the second capillary including the opening.

13. The apparatus of claim 11, wherein the first capillary is made of an optically-transmissive material.

14. The apparatus of claim 11, further comprising a multilayer dielectric coating disposed on the surface of the distal end of the core.

15. The apparatus of claim 11, wherein the first capillary and the second capillary are fixedly coupled.

16. The apparatus of claim 11, wherein the optical fiber includes a buffer layer, the first capillary being fixedly coupled to the buffer layer, the buffer layer of the optical fiber.

17. A method, comprising:
inserting a distal end portion of a first member into a patient's body, the first member having an outer surface that defines a recessed surface, the first member having a second member disposed within the first member configured to redirect laser energy in a lateral direction offset from a longitudinal axis of a distal end portion of the first member; and
after the inserting, activating a laser source to transmit laser energy to the patient's body, the transmitted laser energy passing through the recessed surface of the first member.

18. The method of claim 17, wherein the second member includes a distal end portion of an optical fiber core, a distal end of the optical fiber core having a surface non-perpendicular to the longitudinal axis.

19. The method of claim 17, further comprising adjusting a power level of the laser energy transmitted to the patient's body.

20. The method of claim 17, wherein the inserting the distal end portion of the first member includes inserting the distal end portion of the first member into a urethra.
